(19) United States
(12) Patent Application Publication
(10) Pub. No.: US 2009/0182264 A1
(43) Pub. Date: Jul. 16, 2009

(54) Floating venting and feeding tube for gastro-intestinal feeding

(76) Inventor: Russell J. Eike, Bergenfield, NJ (US)

Correspondence Address: KLAUBER & JACKSON, 411 HACKENSACK AVENUE, HACKENSACK, NJ, 07601, US

(21) Appl. No.: 12/319,611

(22) Filed: Jan. 9, 2009

Related U.S. Application Data
(60) Provisional application No. 61/010,840, filed on Jan. 11, 2008.

(51) Int. Cl. A61M 5/00

(52) U.S. Cl. 604/28; 604/27

(57) ABSTRACT

A venting and feeding tube for use in providing gastrointestinal feeding to patient. The venting and feeding tube has a vent tube associated therewith to vent gases that accumulate in the stomach of the patient. The vent tube has a proximal end adapted to be located external of the patient and a distal end that is adapted to be positioned within the stomach of the patient. The distal end of the vent tube has a floatable device such that the distal end automatically positions itself at the upper area of the stomach where gas accumulates so that the distal end of the vent tube will vent that gas fairly independent of the position or changes in the position of the patient. The floatable device can be an inflatable balloon or material made of a buoyant material.

### BACKGROUND

The present invention relates to a venting and feeding tube for use in feeding a patient, and, more particularly, to a venting and feeding tube having a vent tube with a floating distal end that automatically becomes positioned near the upper region of the patient's stomach.

It is quite common to feed a patient through a gastrostomy tube that passes though the wall of the stomach and which delivers the nutrient directly into the stomach of the patient via the gastrostomy tube.

One of the issues concerned, however with such method of feeding is that as the food is delivered, there is a pocket of gas that forms in the upper area of the stomach and which must be vented for the comfort of the patient. That raises an issue of how to position a vent tube at the location of the pocket of gas and make sure that the venting is being carried out effectively. The problem can be exacerbated by the particular position of the patient, that is, the patient may be in different positions during the feedings or may change position during the feeding and the upper area of the stomach where the pocket of gas is located may, therefore, be in a different location or may change location.

It would therefore be advantageous to have a venting and feeding tube having a vent tube with a distal end that automatically positions itself at the location within the stomach in the upper area thereof where the pocket of gas is located and which would automatically seek that location despite the particular position of the patient or change in position of the patient.

### SUMMARY OF THE INVENTION

Accordingly, the present invention relates to a feeding and venting tube for introducing food into the stomach of a patient and for simultaneous venting of the stomach. The feeding and venting tube includes a flexible, hollow vent tube that has a distal end that is adapted to be introduced into the stomach of the patient to vent the stomach while food is being introduced into the stomach.

The distal end of the vent tube is buoyant or floatable so that, when introduced in to the stomach of the patient, the distal end of the vent tube automatically positions itself at the upper area of the stomach where a pocket of gas normally accumulates.

The buoyancy can be provided by locating an inflatable balloon at the distal end of the vent tube that can be inflated once inside the stomach. Alternatively, the distal end of the vent tube can be comprised of a floatable material or a floatable device attached to or incorporated into that distal end.

The tubing for the vent tube itself is comprised of a thin, flexible material such as silicone so that the distal end of the vent tube can freely rise to the surface of the contents of the stomach.

By the use of a vent tube having a buoyant distal end, the distal end of the vent tube will automatically position itself at the upper area of the patient's stomach where the gas tends to accumulate and is relatively independent of the position of the patient or to changes in the position of the patient during the feeding process.

These and other features and advantages of the present invention will become more readily apparent during the following detailed description taken in conjunction with the drawings herein.

### BRIEF DESCRIPTION OF THE DRAWINGS

FIG. 1 is a side, schematic view of a venting and feeding tube constructed in accordance with the present invention;

FIG. 2 is a side schematic view of the FIG. 1 embodiment with its balloon inflated;

FIG. 3 is a schematic view of a gastrostomy tube feeding system (in dashed lines) delivering formula to the stomach of a patient and including the venting and feeding tube of the present invention; and

FIG. 4 is a schematic view of an alternate exemplary embodiment of a feeding system delivering formula to the stomach of a patient and including the venting tube of the present invention built into a gastrostomy tube.

### DETAILED DESCRIPTION OF THE INVENTION

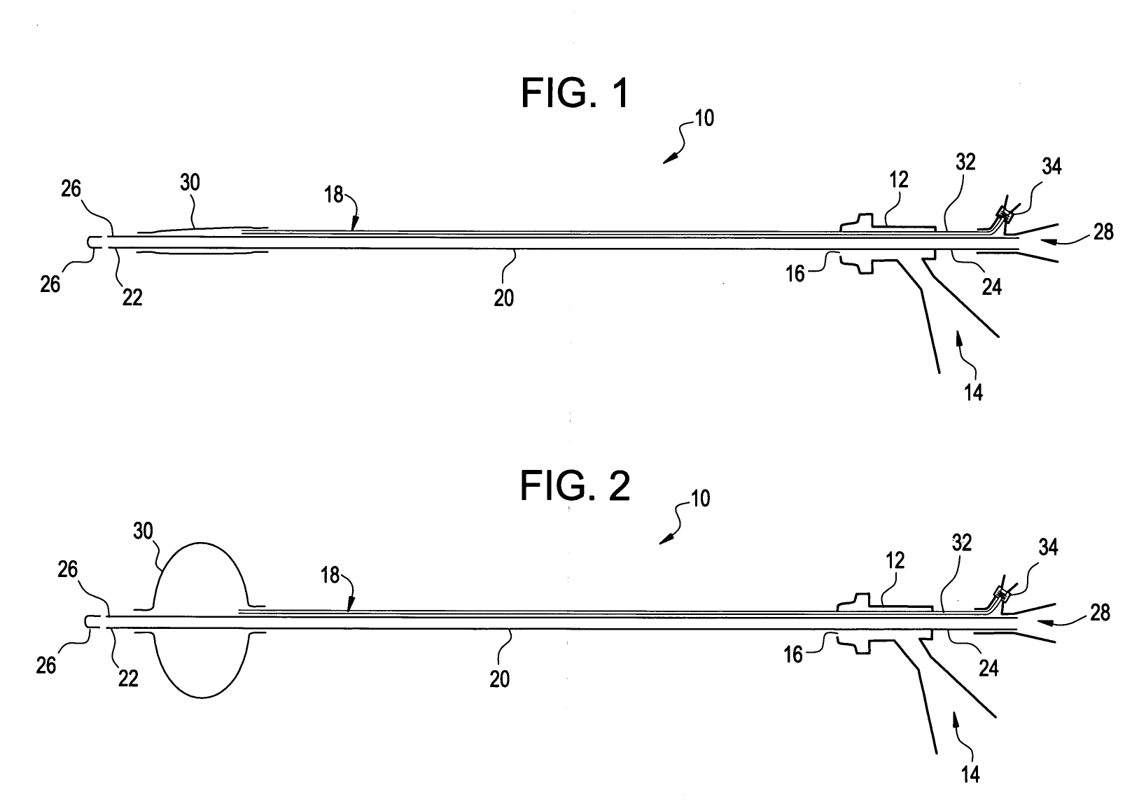

Referring now to FIGS. 1 and 2, there is shown a schematic view of a venting and feeding tube 10 constructed in accordance with the present invention. As can be seen, the venting and feeding tube 10 is used to provide food to the patient. The food, such as a formula, enters through a hub 12 having an inlet 14 and passes inwardly to enter a gastrostomy tube (not shown) via an outlet 16 and through the gastrostomy tube that passes through the wall of the stomach to provide the formula directly into the patient's stomach.

The venting and feeding tube 10 includes a vent tube 18 that comprises thin walled tubing 20 having a distal end 22 and a proximal end 24. The thin walled tubing can be made out of any flexible material, such as silicone or latex, and is generally a hollow tubing having a length of about 5.0 inches and a wall thickness of about 3/32 inches in order to serve the purposes of the present invention in a manner that will become clear. The actual length of the vent tube 18 will vary based on the age and size of the patient and the width must be smaller than the inside diameter of the gastrostomy tube.

There are openings 26 at the distal end 22 of the vent tube 18 to allow gas to pass into and through the tubing 20 to the proximal end 24 where the gas may be vented to the atmosphere through a vent opening 28 or treated in some manner before release to the atmosphere.

As also can be seen, there is an inflatable balloon 30 shown in FIG. 1 in its deflated state and in FIG. 2 in its inflated state. Inflation and deflation of the balloon 30 can be carried out by convention means, including a tube or lumen 32 formed in the vent tube 18 that communicates with the interior of the balloon 30 with an inflating inlet 34 at the proximal end 24 of the vent tube 18. The inflating inlet 34 can include a valve or Luer lock connection to enable the attachment of a syringe to inflate and deflate the balloon 30. As will be seen, the buoyancy of the distal end 22 of the vent tube 18 can be increased by inflating the balloon 30.

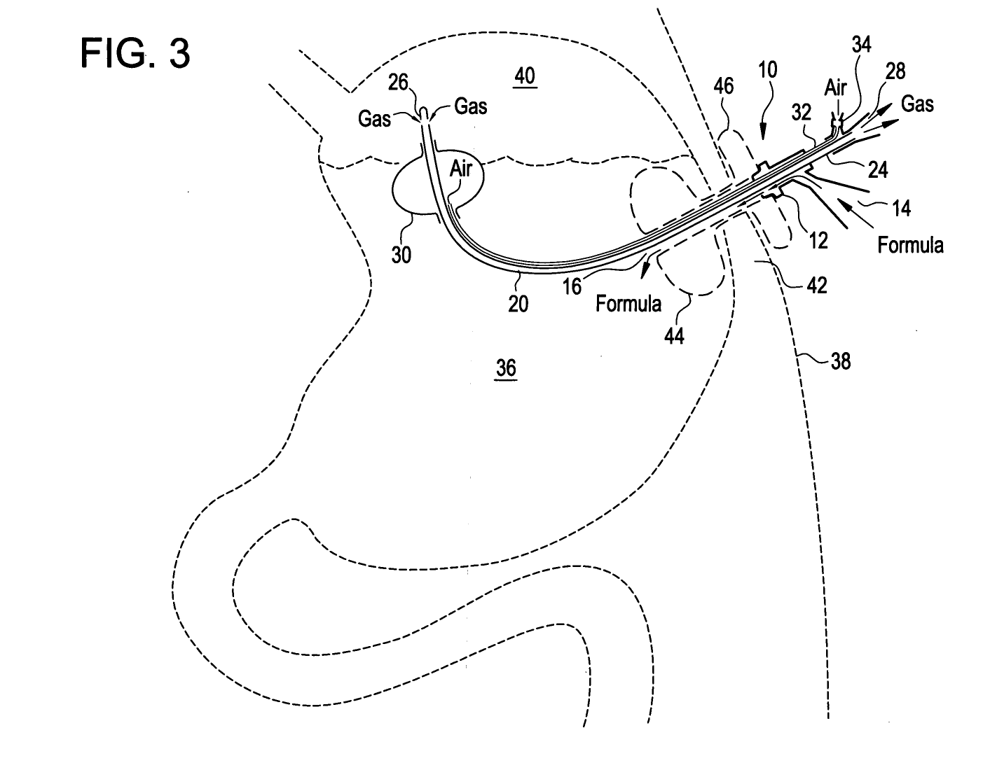

Turning now to FIG. 3, there is shown a schematic view of the venting and feeding tube 10 of the present invention in place within the stomach 36 of a patient 38, having passed through a gastrostomy tube 39, shown in dashed lines, and including a vent tube 18 being used to vent the upper area 40 of the stomach 36 during the feeding of that patient 38. In FIG. 3, it can be seen that the venting and feeding tube 10 enables the formula to pass through the stomach wall 42 to enter the patient's stomach 36 to provide nourishment to the patient 38 while the vent tube 18 simultaneously vents the upper area 40 of the stomach 36.

As also can be seen, the balloon 30 is in its inflated state, thereby giving buoyancy to the distal end 22 of the vent tube 18 and cause the distal end 22 to literally float on the contents of the stomach 36, thereby automatically causing the distal end 22 to locate itself at the upper area 40 of the stomach where the gasses normally accumulate. As such, the gases in that pocket of the upper area 40 can enter through the openings 26 and pass through the vent tube 18 to be discharged through the vent opening 28, thereby relieving the patient 38.

The ability of the distal end 22 of the vent tube 18 to float is determined by the buoyancy of the inflated balloon 30 and is aided by the light and flexible consistency of the tubing 20 that allows the distal end 22 of the vent tube 18 to move upwardly within the stomach 36 by the buoyant effect.

As can therefore be appreciated, the buoyancy causes the distal end 22 of the vent tube 18 to seek the upper surface of the stomach contents and that is true for different positions of the patient and even if the patient changes position during the feeding process.

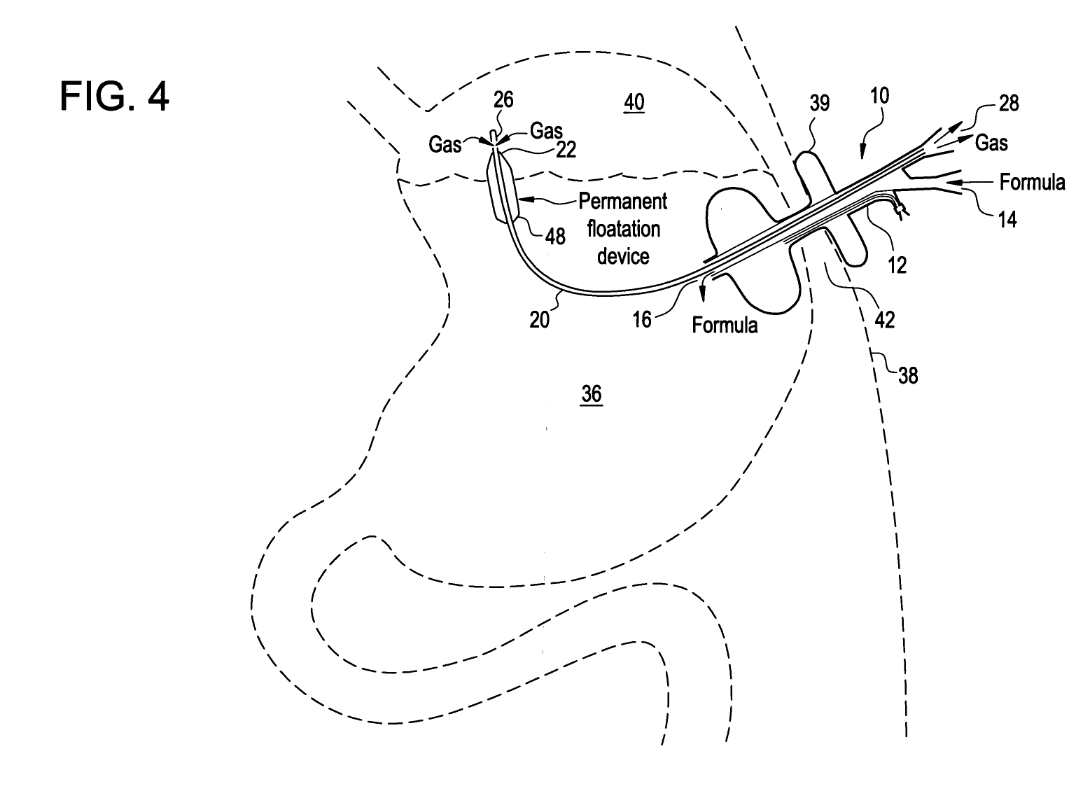

Turning, finally to FIG. 4, there is shown a schematic view of the present invention and like identification numbers are used as in the FIG. 3 embodiment for the same components. In the FIG. 4 embodiment, however, the venting and feeding tube 12 is built into a gastrostomy tube 39. Also, instead of an inflatable balloon at the distal end 22 of the vent tube 18, there is a permanent floatation device 48 that is affixed to the distal end 22 and which is comprised of a floatable material, such as foam plastic. As such, with the FIG. 4 embodiment there is no need to inflate and deflate a balloon since the buoyancy results from the particular material used in the floatation device 48.

Those skilled in the art will readily recognize numerous adaptations and modifications which can be made to the venting and feeding tube and system of the present invention which will result in an improved device and system to allow venting of the stomach during feeding of the patient, yet all of which will fall within the scope and spirit of the present invention as defined in the following claims. Accordingly, the invention is to be limited only by the following claims and their equivalents.

What is claimed is:

1. A venting and feeding tube for use in providing food into the stomach of a patient, the venting and feeding tube having an inlet and outlet for delivering food through the wall of the stomach and having a vent tube comprising a flexible tubing having a distal end adapted to be located in the stomach of a patient and a proximal end adapted to be located exterior of the patient, the distal end being floatable within the stomach so as to automatically find a location at the upper area of the stomach.

2. The venting and feeding tube of claim 1 wherein the flexible tubing is constructed of silicone or latex.

3. The venting and feeding tube of claim 1 wherein the distal end of the flexible tubing has an inflatable balloon that is adapted to be inflated to provide buoyancy thereto.

4. The venting tube of claim 1 wherein the distal end of the flexible tubing is comprised of a buoyant material.

5. A method of venting the stomach of a patient during gastric feeding, the method comprising the steps of:
introducing a venting and feeding tube into the stomach of a patient for providing food to a patient;
introducing a vent tube into the stomach of a patient, the vent tube having a floatable distal end,
allowing the distal end of the vent tube to position itself in the upper area of the stomach, and
venting gas located at the upper area of the stomach to the atmosphere.

6. A system for feeding a patient comprising:
a venting and feeding tube for providing nourishment directly to the stomach of a patient;
a vent tube for venting the stomach of a patient, the vent tube being adapted to be introduced into the stomach of a patient, the vent tube comprising a thin flexible tubing having a buoyant distal end adapted to be located within the stomach of a patient and a proximal end adapted to be located external of a patient.
